(19) United States
(12) Patent Application Publication
(10) Pub. No.: US 2020/0229447 A1
(43) Pub. Date: Jul. 23, 2020

(54) PRESERVATIVE COMPOSITION

(71) Applicant: King Bio, Inc., Asheville, NC (US)

(72) Inventor: Frank J. King, Leicester, NC (US)

(21) Appl. No.: 16/838,661

(22) Filed: Apr. 2, 2020

Related U.S. Application Data
(63) Continuation of application No. 15/876,301, filed on Jan. 22, 2018.
(60) Provisional application No. 62/458,823, filed on Feb. 14, 2017.

(51) Int. Cl. A01N 65/08; A61K 8/88; A01N 37/18; A61K 31/785; A61Q 19/00; A01N 65/36; A61K 36/752; A61K 36/52; A61K 36/355; A23L 3/3526; A61K 8/9789; A23L 3/3472

(52) U.S. Cl. CPC A01N 65/08; A61K 8/88; A01N 37/18; A61K 31/785; A61Q 19/00; A01N 65/36; A61Q 11/00; A61K 36/52; A61K 36/355; A23L 3/3526; A61K 8/9789; A23L 3/3472; A61K 36/752

(57) ABSTRACT

A preservative composition is provided and includes polylysine, shagbark hickory bark extract, honeysuckle and citric extract(s).

### CROSS-REFERENCE TO RELATED APPLICATION

This application is a continuation of U.S. application Ser. No. 15/876,301 filed Jan. 22, 2018, which claims priority to U.S. Provisional Application Ser. No. 62/458,823, filed Feb. 14, 2017, the disclosures of which are incorporated by reference in their entireties.

### FIELD AND BACKGROUND OF THE INVENTION

The present invention relates to a natural preservative, and more particularly to a natural preservative that extends shelf life and that provides antimicrobial properties during the processing, preparation, packaging, and use of foodstuff, nutraceuticals, cosmetics, pharmaceuticals and the like. Such compositions are susceptible to microorganisms which may make the compositions unsuitable for consumption or use. In the past such compositions often utilize potassium sorbate and/or parabens as a preservative. There is, however, a desire to eliminate the use of potassium sorbate and parabens due to there being health concerns with their use.

### SUMMARY OF THE INVENTION

To this end, it is desirable to provide a natural preservative composition that when used as an additive extends shelf life and provides antimicrobial properties to a wide variety of products. Additionally, the natural preservative includes ingredients that are GRAS grade materials are substantially flavor, smell and color free. The present invention provides a natural preservative composition comprising polylysine, shagbark hickory bark extract, honeysuckle extract and citrus extract(s). In one embodiment, the natural preservative is low concentration as compared to the product to which added, comprises 0.005 to 0.50 percent polylysine, 0.001 to 0.005 percent citrus extract, 0.15 to 0.25 percent shagbark hickory bark extract and 0.10 to 0.50 percent honeysuckle extract.

### DETAILED DESCRIPTION OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items.

The term “about,” as used herein when referring to a measurable value such as an amount of a compound, dose, time, temperature, and the like, is meant to encompass variations of 20%, 10%, 5%, 1%, 0.5%, or even 0.1% of the specified amount. Unless otherwise defined, all terms, including technical and scientific terms used in the description, have the same meaning as commonly understood by one of ordinary skill in the art to which this invention belongs.

As used herein, the terms “comprise,” “comprises,” “comprising,” “include,” “includes” and “including” specify the presence of stated features, integers, steps, operations, elements, and/or components, but do not preclude the presence or addition of one or more other features, integers, steps, operations, elements, components, and/or groups thereof.

As used herein, the term “consists essentially of” (and grammatical variants thereof), as applied to the compositions and methods of the present invention, means that the compositions/methods may contain additional components so long as the additional components do not materially alter the composition/method. The term “materially alter,” as applied to a composition/method, refers to an increase or decrease in the effectiveness of the composition/method of at least about 20% or more.

All patents, patent applications and publications referred to herein are incorporated by reference in their entirety. In case of a conflict in terminology, the present specification is controlling.

The present invention provides a natural preservative for application to limit microbial activity, provide antioxidant properties, and improve shelf life without an adverse affect on the characteristics of the product such as flavor, smell or color. The term “natural preservative” is used herein as intended to mean components derived from GRAS grade materials wherein the combination of components provide a preservative effect against the static or reduce growth of microbes, i.e., to provide an “antimicrobial” effect. Exemplary products to which the natural preservative composition of the invention may be added to include foodstuffs, nutraceuticals, pharmaceuticals, cosmetics, toothpaste and the like. The term “foodstuff” is intended to mean a substance which is suitable for human or animal consumption, and includes dairy products, animal foods, snack foods, sauces and gravies, soups, casseroles, juices, prepared meat and meat spreads, cereals, margarine, salad dressings, condiments (e.g., ketchup and mustard), meat, fish and shellfish, and poultry. Nutraceuticals include homeopathic and herb-based remedies. Cosmetics include makeup, moisturizers, hair care products, shaving products and the like.

The natural preservative composition of the invention comprises polylysine, shagbark hickory bark extract, honeysuckle extract and citric extracts. In one embodiment, the natural preservative comprises 0.005 to 0.50 percent polylysine, 0.001 to 0.005 percent citrus extract, 0.15 to 0.25 percent shagbark hickory bark extract and 0.10 to 0.50 percent honeysuckle extract.

Exemplary citrus extracts may include orange extract, lime extract, and lemon extract, alone or in combination.

The natural preservative may include other GRAS additives to improve flavor, smell and/or color characteristics of the natural preservative composition. For example, 0.0001 to 0.05 percent of stevia may be added to mask flavor.

The present invention will be further illustrated by the following non-limiting examples.

### EXAMPLES

### Control

The Asthma Care® product sold by King Bio, Inc., Asheville, N.C. was provided:

| Component | % |
|---|---|
| Water and Actives | 99.8925 |
| Potassium Sorbate | 0.1000 |
| Citric Acid | 0.0075 |

### Example 1

The Asthma Care® product of the Control was provided without the potassium sorbate and citric acid as follows:

| Component | % |
|---|---|
| Water and Actives | 99.1300 |
| Polylysine | 0.0140 |
| Shagbark Hickory | 0.270 |
| Burk Extract | |
| Honeysuckle Extract | 0.540 |
| Citrus Extract | 0.0430 |
| Stevia | 0.0012 |

### Example 2

| Component | % |
|---|---|
| Water and Actives | 99.3900 |
| Shagbark Hickory | 0.0100 |
| Burk Extract | |
| Honeysuckle Extract | 0.3800 |
| Citrus Extract | 0.0300 |
| Stevia | 0.00084 |

Twenty-eight day accelerated stability tests were performed on the Control and Examples 1 and 2 for a number of microbes according to the USPTO. Antimicrobial Effectiveness Testing Chapter <51>.

CONTROL
Day 9 Plating

| | E. coli | S. aureus | P. aeruginosa | C. albicans | A. brasiliensis |
|---|---|---|---|---|---|
| log 10 | <2.00 | <2.00 | 3.66 | 5.53 | 4.17 |
| Log Reduction | >3.72 | >3.59 | 1.24 | 0.15 | 1.01 |

Day 14 Plating

| | E. coli | S. aureus | P. aeruginosa | C. albicans | A. brasiliensis |
|---|---|---|---|---|---|
| | cfu/ml. | cfu/ml. | cfu/ml. | cfu/ml. | cfu/ml. |
| log 10 | <2.00 | <2.00 | <2.00 | >5.53 | 4.09 |
| Log Reduction | >3.72 | >3.59 | >2.90 | <0.05 | 1.09 |
| Pass/Fail | Pass | Pass | Pass | Fail | Pass |

Day 28 Plating

| | | | | | |
|---|---|---|---|---|---|
| log 10 | <2.00 | <2.00 | <2.00 | >5.59 | >4.18 |
| Log Reduction | >3.72 | >3.59 | >2.90 | <0.09 | <1.00 |
| Pass/Fail | Pass | Pass | Pass | Fail | Fail |

As can be seen, the Control failed a number of the tests.

### Example 1

Day 9 Plating

| | E. coli | S. aureus | P. aeruginosa | C. albicans | A. brasiliensis |
|---|---|---|---|---|---|
| log 10 | <2.00 | <2.00 | <2.00 | <2.00 | 3.83 |
| Log Reduction | >3.72 | >3.59 | >2.90 | >3.68 | 1.35 |

Day 14 Plating

| | E. coli | S. aureus | P. aeruginosa | C. albicans | A. brasiliensis |
|---|---|---|---|---|---|
| | cfu/ml. | cfu/ml. | cfu/ml. | cfu/ml. | cfu/ml. |
| log 10 | <2.00 | <2.00 | <2.00 | <2.00 | 3.72 |
| Log Reduction | >3.72 | >3.59 | >2.90 | >3.68 | 1.46 |
| Pass/Fail | Pass | Pass | Pass | Pass | Pass |

Day 28 Plating

| | | | | | |
|---|---|---|---|---|---|
| log 10 | <2.00 | <2.00 | <2.00 | <2.00 | >4.18 |
| Log Reduction | >3.72 | >3.59 | >2.90 | >3.68 | >1.00 |
| Pass/Fail | Pass | Pass | Pass | Pass | Pass |

This demonstrates the antimicrobial effect of the natural preservative composition of Example 1.

### Example 2

Day 9 Plating

| | E. coli | S. aureus | P. aeruginosa | C. albicans | A. brasiliensis |
|---|---|---|---|---|---|
| log 10 | <2.00 | <2.00 | <2.00 | <2.00 | 3.86 |
| Log Reduction | >3.72 | >3.59 | >2.90 | >3.68 | 1.32 |

Day 14 Plating

| | E. coli | S. aureus | P. aeruginosa | C. albicans | A. brasiliensis |
|---|---|---|---|---|---|
| | cfu/ml. | cfu/ml. | cfu/ml. | cfu/ml. | cfu/ml. |
| log 10 | <2.00 | <2.00 | <2.00 | <2.00 | 3.95 |
| Log Reduction | >3.72 | >3.59 | >2.90 | >3.68 | 1.23 |
| Pass/Fail | Pass | Pass | Pass | Pass | Pass |

Day 28 Plating

| | | | | | |
|---|---|---|---|---|---|
| log 10 | <2.00 | <2.00 | <2.00 | <2.00 | >3.92 |
| Log Reduction | >3.72 | >3.59 | >2.90 | >3.68 | >1.26 |
| Pass/Fail | Pass | Pass | Pass | Pass | Pass |

This demonstrates the antimicrobial effect of the natural preservative composition of Example 2.

Having thus described certain embodiments of the present invention, it is to be understood that the invention defined by the appended claims is not to be limited by particular details set forth in the above description including the examples as many apparent variations thereof are possible without departing from the spirit or scope thereof as hereinafter claimed.

That which is claimed:

1. A preservative for providing antimicrobial properties comprising polylysine, citrus extract, shagbark hickory extract and honeysuckle extract.

2. The preservative of claim 1 in combination with a foodstuff, nutraceutical, pharmaceutical, cosmetic or toothpaste.
